(19) United States
(12) Patent Application Publication
(10) Pub. No.: US 2024/0298947 A1
(43) Pub. Date: Sep. 12, 2024

(54) A DIAGNOSTIC DEVICE AND METHOD OF DIAGNOSIS THEREOF FOR KIDNEY FUNCTIONING

(71) Applicant: OMINAR INNOVATIONS PRIVATE LIMITED, JALGAON (IN)

(72) Inventors: VISHAL ANANDRAO SALUNKHE, JALGAON (IN); AMAR SUNIL MORE, PUNE (IN)

(73) Assignee: OMINAR INNOVATIONS PRIVATE LIMITED, JALGAON, MH (IN)

(21) Appl. No.: 18/039,256

(22) PCT Filed: Nov. 30, 2021
(86) PCT No.: PCT/IN2021/051123

§ 371 Date: May 29, 2023

(30) Foreign Application Priority Data
Nov. 30, 2020 (IN) 202021052074

(51) Int. Cl. A61B 5/20; G01N 21/78; G16H 10/40

(52) U.S. Cl. CPC A61B 5/201; A61B 5/207; G01N 21/78; G16H 10/40

(57) ABSTRACT

The present invention relates to a diagnostic device and method for diagnosis thereof for kidney functioning configured for diagnosing one or more ailments associated with the kidneys of a human being. The device comprising of a housing (10A) and a tray (10B). The housing (10A) forms the outer cover in which the tray (10B) is placed. The housing (10A) has a plurality of at least six cavities configured thereon. The tray (10B) is a filter paper printed with a hydrophobic pattern. The device is with the image capturing and image processing unit to process the image to identify the change of colour in reaction zones due to specific biomarkers such as total proteins, creatinine, uric acid, nitrite test, leukocyte esterase (LE), and phosphates. The device is with data interpretation unit to compute the quantitative values.

### FIELD OF THE INVENTION

The present invention relates to digital healthcare diagnostic devices. The present invention more particularly relates to a diagnostic device and method for diagnosis thereof for kidney functioning which is configured for diagnosing one or more ailments associated with the kidneys of a human being.

### BACKGROUND OF THE INVENTION

Kidney failure is without any obvious symptoms. Diabetes, hypertension, urinary tract infection and kidney stones usually lead to kidney failure. Almost twenty crores Indians are suffering from chronic kidney disease (CKD). CKD progresses slowly from stage 1 (mild damage) to stage 5 (failure). Usually, blood and urine tests help doctors to understand the overall functioning of the kidney and possible progress of the disease. A diagnosis is made based on severe clinical symptoms or by chance findings from screening tests such as dipsticks or lab-based blood tests (Webster et al., Lancet, 2017). There is an unmet need for early diagnosis of kidney disease. If the person has developed clinical symptoms, the kidney disease has progressed to an advanced stage or to a point where they must have regular dialysis or receive a kidney transplant to stay alive. The kidney damage is essentially result of combination of or any of CKD, chronic urinary tract infections or kidney stones.

The present medical demand for point of care device is to detect kidney stones as kidney stone disease affects 20% of general population worldwide (PMID 29321449). In India, around 12% populations have urinary stones out of which 50% end up with kidney dysfunction (PMID 29515627). The urinary tract infections are also the reason for kidney damage if left untreated which affects around 150 million people worldwide (PMID 27692880). The screening, and monitoring of kidney's health is missing for quick intervention by patient or clinician. Most clinicians prefer to collect 24-hour urine or early morning urine of the patient however it is not feasible practically.

CN210953827U discloses an acute kidney injury short-term test kit and a method of identifying urinary tract infection in children of two years old and younger based on using a reagent containing nitrotetrazolium blue an infection of the urinary tract of the child following a change in colour from yellow to blue-violet.

CN110473167 discloses a medical image processing method for a urinary sediment image recognition system and a method based on deep learning.

US20190183400 discloses a cognitive algorithm which is executed by an image processor for comparing images of normal urinary tract segments to the captured images to detect abnormalities in the urinary tract and provide an indication of the abnormalities to a user.

However, inventions disclosed until now suffer from disadvantage that in rural settings the cost involved for the travel of patients and overheads are usually equal or more than the actual pathology tests, which leads to either inflated diagnostics bills or avoiding crucial screening leading to disease aggravation.

The affordable point of care devices for routine pathology such as home-based kidney screening device is an urgent and unmet need to provide faster, sensitive and quantitative diagnostic or screening report without any complications. The spectrophotometer is commonly used in any commercial pathology laboratory and its function is based on the principle of the absorbance or transmittance. Therefore, there is a need for an alternative home-based diagnostic device and simple, yet effective method for diagnosis of chronic kidney disease that alleviates the aforementioned drawbacks.

### OBJECT OF THE INVENTION

The main object of the present invention is to provide a diagnostic device and method for diagnosis thereof for kidney functioning which is configured for diagnosing one or more ailments associated with the kidneys of a human being.

Another object of the present invention is to provide a urine sampling device comprising a housing and a tray for predicting the possible health condition of the kidney.

Yet another object of the present invention is to provide a method for identifying and quantifying the bio marker and generating a corresponding report for predicting the possible health condition of the kidney.

Yet another object of the present invention is to provide a method comprising the steps of processing the captured images to compute the quantitative values from the raw values depending upon the intensity of the colour of the designed reaction zones for predicting the possible health condition of the kidney.

Yet another object of the present invention is to provide a clinical range protein standard curve using pooled samples from hospitals to discriminate between healthy and sick individuals using their urine samples.

Still another object of the present invention is to provide a report for the status of kidney functioning to the public at a reduced cost and at a substantially reduced amount of turnaround time to provide a home-based kidney screening device.

### SUMMARY OF THE INVENTION

The present invention relates to a diagnostic device and method for diagnosis thereof for kidney functioning which is configured for diagnosing one or more ailments associated with the kidneys of a human being.

In a preferred embodiment, the present invention provides a urine sampling device comprising a housing and a tray for predicting the possible health condition of the kidney. The tray is a filter paper printed with a hydrophobic pattern to limit the water or other chemicals to flow out of designated reaction zone. The designated reaction zones are the portions that align with the plurality of cavities formed on the housing. Once the user deposits the urine sample into the single use device, appropriate reactions take place at each designated reaction zone, and if user tests positive for any of the biomarker, a colour change takes place on the corresponding designated reaction zone which is captured as an image of the same by a smartphone having a camera. An image processing is performed by processing module to process the images of the designated reaction zones for colour detection for six biomarkers. The data pre-processing is performed for processing image and picking colour code (in the form of raw values) for biomarkers such as total proteins, creatinine, uric acid, nitrite test, leukocyte esterase (LE), and phosphates.

In another embodiment, the present invention provides a method for identifying and quantifying the bio marker and generating a corresponding report for predicting the possible health condition of the kidney.

In still another embodiment, the present invention provides a clinical range protein standard curve using pooled samples from hospitals to discriminate between healthy and sick individuals using their urine samples.

The present invention provides a report of the routine kidney health which is made available to the public at a reduced cost relative to the cost of having to go to the diagnostic centre, using the device and method of the present invention. Furthermore, the results provided at a substantially reduced amount of turnaround time, as compared to 24 to 48 hours that otherwise the user or patient has to wait using the conventional methods.

### BRIEF DESCRIPTION OF DRAWINGS

Objects, features, and advantages of the invention will best be understood from the following description of various embodiments thereof, selected for the purposes of illustration, and shown in the accompanying drawings, in which:

FIG. 1 illustrates a schematic view of a housing of a diagnostic device, in accordance with an embodiment of the present invention;

FIG. 2 illustrates a schematic view of a tray of a diagnostic device, in accordance with an embodiment of the present invention.

FIG. 3 is a schematic view of depicting the steps involved in performing a self diagnosis of a urine sample using a diagnostic device and a method in accordance with an embodiment of the present invention.

FIG. 4 is a representation of a kidney screening device.

FIG. 5 is an illustrative view of taking picture of the device after reactions are developed in the wells.

FIG. 6(a), FIG. 6(b), FIG. 6(c) and FIG. 6(d) are a representation of total protein and creatinine analysis (a) protein test on diagnostic paper platform; (b) creatinine test on diagnostic paper platform; (c) graph suggesting colour intensity values vs. concentration of total proteins (10 to 4000 mg/L) using image J software; and (d) graph suggesting colour intensity values vs. concentration of creatinine (10 to 180 mg/dl) using image J software.

### DETAILED DESCRIPTION OF THE INVENTION

The invention will now be described in detail in connection with certain preferred and optional embodiments, so that various aspects thereof may be more fully understood and appreciated.

The present invention relates to a diagnostic device configured for diagnosis of one or more ailments associated with the kidneys of a human being and a method for diagnosis thereof.

In a preferred embodiment, the present invention provides a urine sampling device comprising a housing and a tray. The tray is a filter paper printed with a hydrophobic pattern to limit the water or other chemicals to flow out of designated reaction zone. The designated reaction zones are the portions that align with the plurality of cavities formed on the housing. Once the user deposits the urine sample into the single use device, appropriate reactions take place at each designated reaction zone, and if user tests positive for any of the biomarker, a colour change takes place on the corresponding designated reaction zone which is captured as an image of the same by a smartphone having a camera. An image processing is performed by processing module to process the images of the designated reaction zones for colour detection for six biomarkers. The data pre-processing is performed for processing image and picking colour code (in the form of raw values) for biomarkers such as total proteins, creatinine, uric acid, nitrite test, leukocyte esterase (LE), and phosphates. The method further comprises the steps of processing captured images to compute the quantitative values from the raw values depending upon the intensity of the colour of the designed reaction zones. The data interpretation is done using artificial intelligence (AI) and at least one machine learning (ML) algorithms. The method further comprises the step of displaying the results in the form of a report for the possible health condition of the kidney. Therefore, the report of the routine kidney health is made available to the public at a reduced cost than the diagnostic centre.

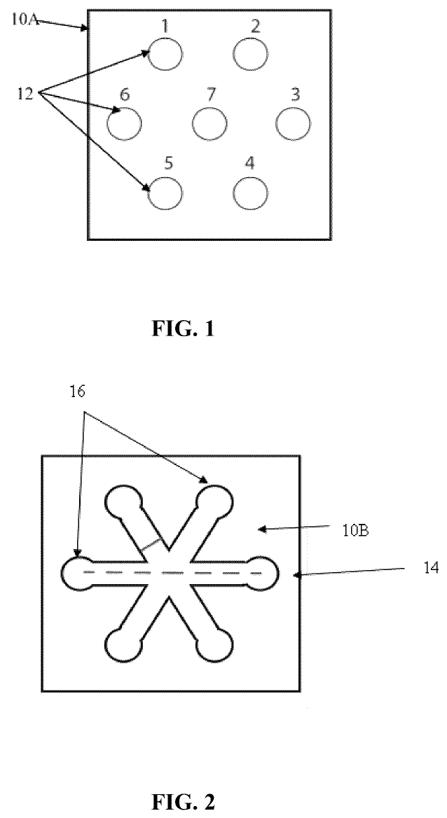

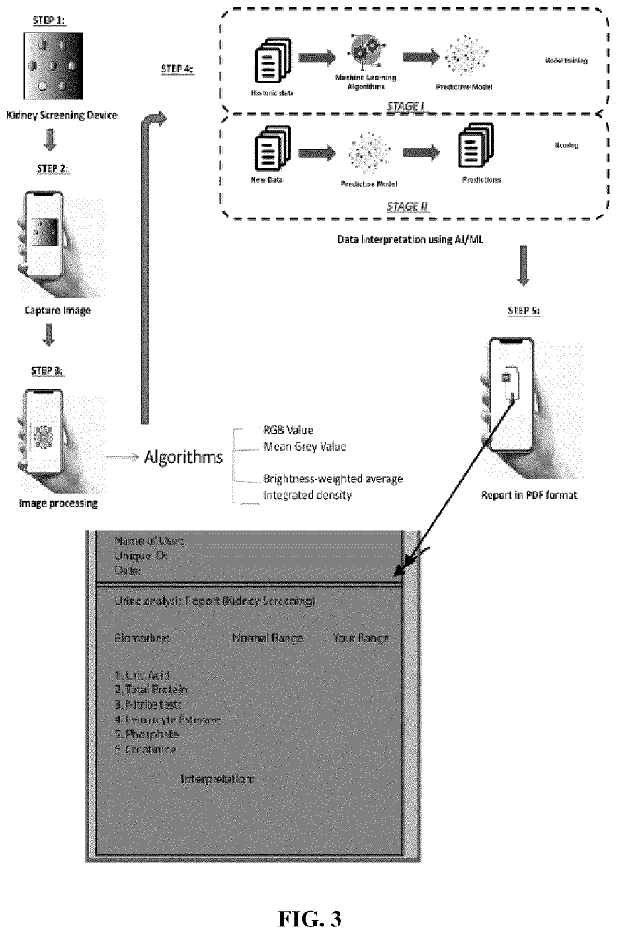

FIG. 1 and FIG. 2 illustrate schematic views of elements of a diagnostic device 10, in accordance with an embodiment of the present invention. The diagnostic device (10) comprising of; a housing (10A) and a tray (10B). The housing (10A) forms the outer cover in which the tray (10B) is placed, in accordance with one embodiment. More specifically, the housing (10A) resembles a cassette like structure that accommodates the tray (10B) there within. The housing (10A) has a plurality of cavities including at least six cavities configured thereon. In accordance with an embodiment of the present invention, the tray 10B is a filter paper printed with a hydrophobic pattern, as illustrated in FIG. 2. The purpose of creating hydrophobic barriers via the hydrophobic pattern (14) is to limit the water or other chemicals to flow out of designated reaction zone (16). More specifically, the designated reaction zones (16) are the portions that align with the plurality of cavities (12) formed on the housing (10A).

The central cavity of the plurality of cavities (12) is the main sample receiving cavity, however the sample is also placed directly in reaction cavity. The sample deposited on the tray (10B) at the area overlapping with the central cavity makes its way to the designated reaction zones (16) owing to presence of the hydrophobic pattern (14). The purpose of depositing the sample is to identify and quantify the biomarkers present in the sample. The identification of each biomarker is performed by colorimetric reactions including but not limited to colour reactions and each reaction is unique and specific to the biomarker.

The chemicals required to identify the biomarkers, which are conventionally known in the art, are provided on the designated reaction zones (16) in appropriate concentration and volume on tray (10B) and are dried up for some time. Each designated reaction zone (16) corresponds to a specific biomarker. It is to be noted that the diagnostic device is a single use device. Once the user deposits the urine sample into the single use device, appropriate reactions take place at each designated reaction zone 16, and if user tests positive for any of the biomarker, a colour change takes place on the corresponding designated reaction zone.

The housing (10A) in the present invention, is shown to have six cavities for measuring six bio markers in a urine sample. However, in the preferred embodiment, the number of cavities is two for total protein and creatinine. The housing (10A) is having more or a smaller number of cavities depending on the biomarkers required to be tested. Also, there can be desirable variations in the design and number of cavities, tray, etc.

The diagnostic device (10) is with housing (10A) having plurality of cavities with at least six cavities and a tray having hydrophobic pattern. The designated reaction zones aligning with plurality of cavities. The diagnostic device (10) is with an image capturing and image processing unit to process the image to identify the change of colour in reaction zones due to specific biomarkers including but not limited to total proteins, creatinine, uric acid, nitrite test, leukocyte esterase (LE), red blood cells (RBCs), glucose, pus cells, and phosphates. In the preferred embodiment of the present invention, total proteins and creatinine biomarkers are focused. The diagnostic device (10A) is with data interpretation unit to compute the quantitative values depending upon the intensity of colour of designated reaction zones. The dimensions of the diagnostic device are selected from the following dimensions:
Length of the device=5 cm to 10 cm;
Width of the device=2.5 cm to 5 cm;
Height of the device=0.4 cm to 2 cm;
Diameter of each well=1.5 cm to 3 cm; and
Diameter of the inner hydrophobic wall=1.35 cm to 5 cm

FIG. 3 illustrates the method steps for identifying and quantifying the biomarker, and generating a corresponding report, in accordance with an embodiment of the present invention. Referring to FIG. 3, once the colour change has taken place at the designated reaction zone (16), the method comprises the step of user capturing an image of the same. For the sake of convenience, in accordance with an embodiment of the present invention, the step of capturing the image of the designated reaction zones (16) is facilitated by a smartphone having a camera.

Once the image has been captured, the method comprises the step of performing image processing of the captured image. In accordance with an embodiment of the present invention, the image processing is performed via a processing module that is specifically designed to process the images of the designated reaction zones (16). In an embodiment, the image processing is performed via the smartphone. The processing module required for processing the image is provided on the smartphone in the form of an application. An advantageous aspect of this application is that this application may be considered to be an alternative to the traditional spectrophotometer. However, the application or the system, in accordance with at least one embodiment of the present invention, may be based on the principle of the reflectance of the light. Based on the preliminary experiments, it has been found that using system in accordance with at least one embodiment of the present invention, the raw intensity values are directly proportional to the concentration of the biomarker.

The step of image processing includes image processing and colour detection for at least six biomarkers. The colour/s at the designated reaction zones is captured by the smartphone as input data. The data pre-processing is nothing but processing image and picking colour code (in the form of raw values) for biomarkers such as total proteins, creatinine, uric acid, nitrite test, leukocyte esterase (LE), and phosphates. The processing happens through both online and off-line mode.

The clinical range protein standard curve using pooled samples from hospitals showing protein concentration from 10 mg/L to 4000 mg/L and concentration of creatinine from 10 to 180 mg/dl, clearly visible and reproducible and helps to discriminate between healthy and sick individuals using their urine samples with a sensitivity and specificity of 85-90% and an accuracy of 80-99% using the diagnostic device of the present invention.

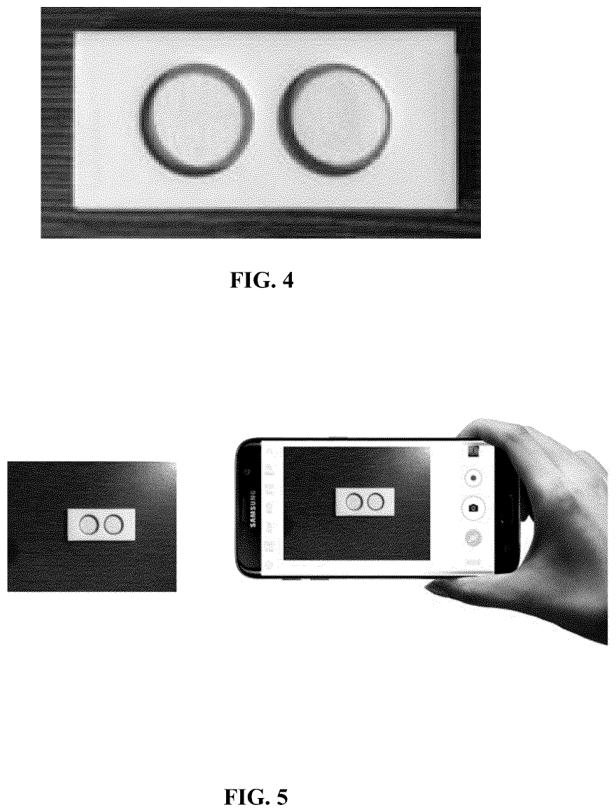

FIG. 4 is a representation of a kidney screening device.

FIG. 5 is an illustrative view of taking picture of the device after reactions are developed in the wells.

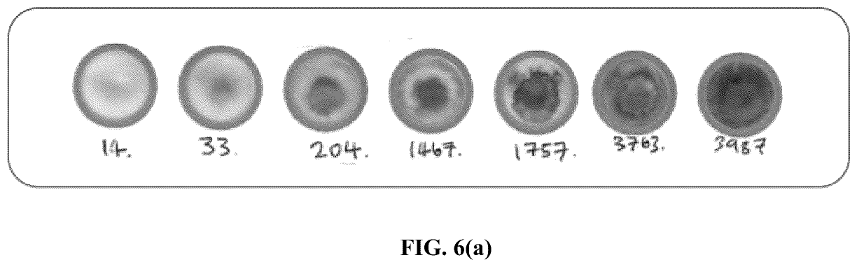

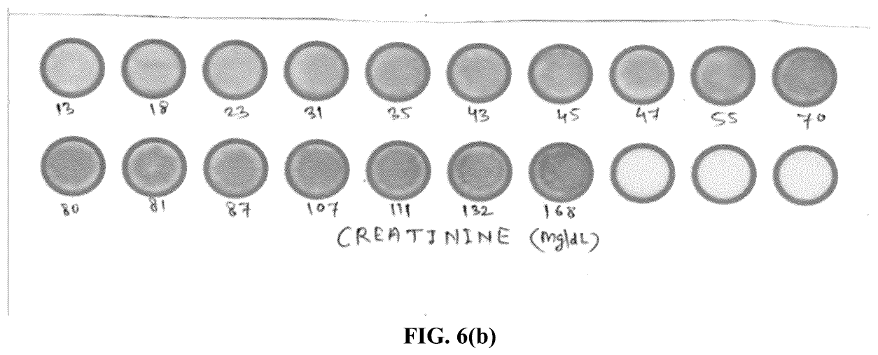

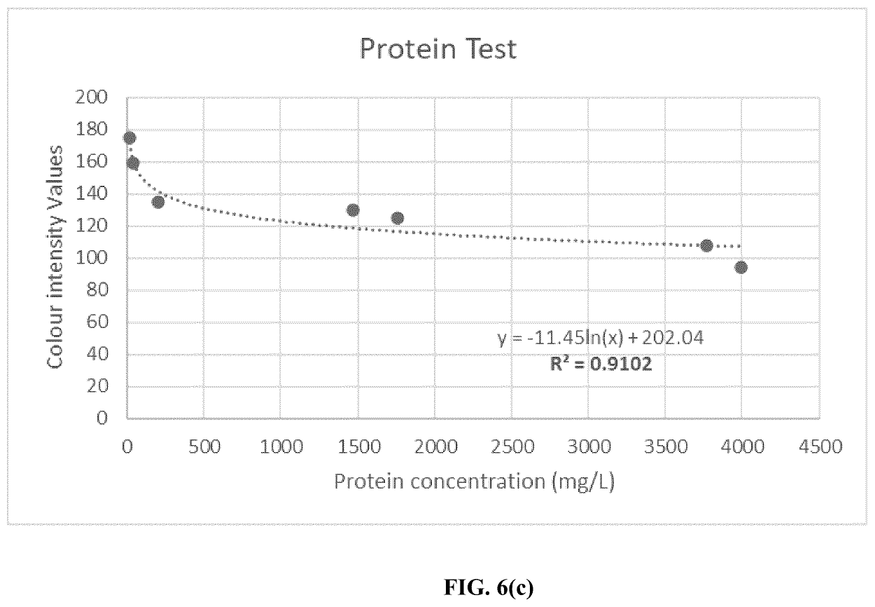

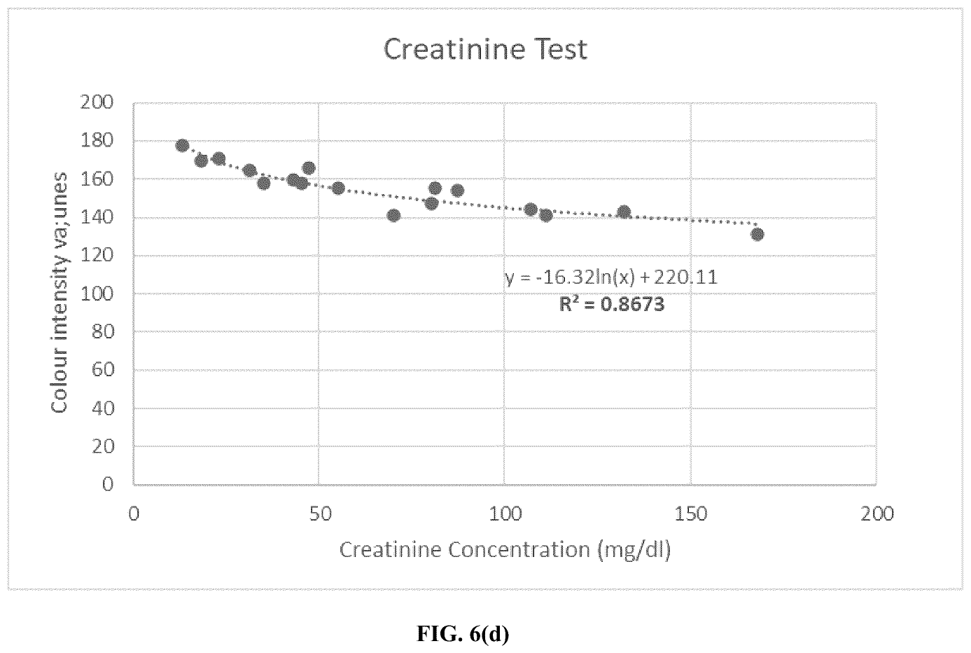

FIG. 6 is a representation of total protein and creatinine analysis (a) protein test on diagnostic paper platform; (b) creatinine test on diagnostic paper platform; (c) graph suggesting colour intensity values vs. concentration of total proteins (10 to 4000 mg/L) using image J software; and (d) graph suggesting colour intensity values vs. concentration of creatinine (10 to 180 mg/dl) using image J software.

Testing for protein is based on the phenomenon called the “Protein Error of Indicators” (ability of protein to alter the color of some acid-base indicators without altering the pH). In a solution void of protein, tetrabromphenol blue, buffered at a pH of 3, is yellow. However, in the presence of protein (albumin), the color changes to green, then blue, depending upon the concentration. The colour patterns of this test clearly discriminate between healthy individuals and dialysis patients. These colour patterns are reproducible and are ready to use to feed into to the software system of the present diagnostic device for developing training models of artificial intelligence (AI) and machine learning (ML) system.

In yet another embodiment, the present invention provides a method comprising the steps of processing the captured images to compute the quantitative values from the raw values depending upon the intensity of the colour of the designed reaction zones (16). More specifically, data collected during clinical trials will be used as historical data/existing data. In future, the captured images will then be compared to the existing data and possible kidney health condition outcomes are classified in “classes”, which is nothing but dependent variable which varies based on combinations of input variables.

It is to be noted that at least one AI and ML algorithms for predicting classes including but not limited to logistic regression, decision tree, neural network, and random forest is used for computing the classes. More specifically, a cloud-based server stores the existing data, and new data is added to the server as and when the device and the method of the present invention are used, and on this continually collecting data, any one or more of the aforementioned machine learning algorithms is employed for computing the class indicating the possible health condition of kidney. The data interpretation is done using AI and at least one ML algorithms.

The method further comprises the step of displaying the possible health condition of the kidney in the form of a report. This interpretation will help the user to understand the individual status of its kidney's health and whether or not, the individual should visit nearest general physician or nephrologist. The kidney screening device does not provide any prescription, treatment or medications. The user always has to see his nearest general physician or nephrologist. In another embodiment, the report is saved by the user in a PDF format. In yet another embodiment, the report is made available to the user on the smartphone application.

Therefore, the report of the routine kidney health is made available to the public at a reduced cost relative to the cost of having to go to the diagnostic centre, using the device and method of the present invention. Furthermore, the results provided at a substantially reduced amount of turnaround time, as compared to 24 to 48 hours that otherwise the user/patient has to wait using the conventional methods.

In yet another embodiment, the device and method of the present invention has been designed to test a urine sample for a routine kidney health test. However, it is to be noted that the device and method are not restricted to being applicable for a routine kidney health test. Rather a kidney health test is one of the applications of the device and method as described in the present invention. The said device and method are further used to test liver, heart, and any other organ function using specific set of biomarkers, not only in human samples but also from any animal species samples. Each species and organ require specific calibration with reference to the biomarkers and its level.

The device and method, as disclosed in the present invention, is also configured to perform different kinds of blood tests. The remote cloud server, in accordance with one implementation, is fed with clinical data available for different kinds of blood test as reference data to which the blood samples is compared. The device and the method are further configured to employ one or more machine learning models to improve on the dataset for providing quicker results to the user. The mobile application is universal read out system and is used for detection of any biomarker from any initial sample material such as water, urine, blood, sputum, cerebrospinal fluid, bone marrow, milk etc. or any cancer biomarkers is detected.

As will be readily apparent to those skilled in the art, the present invention may easily be produced in other specific forms without departing from its essential characteristics. The present embodiments are, therefore, to be considered as merely illustrative and not restrictive, the scope of the invention being indicated by the foregoing description, and all changes which come within therefore intended to be embraced therein.

What is claimed is:

1. A diagnostic device (10) comprising:
a housing (10A); and
a tray (10B);
wherein,
the housing (10A) forms an outer cover in which the tray (10B) is placed wherein the housing (10A) has a plurality of cavities (12) that forms a cassette like structure of the housing (10A);
the tray (10B) is a filter paper printed with a hydrophobic pattern (14) which forms hydrophobic barrier that limits water or other chemicals flow out of a designated reaction zone (16);
the designated reaction zones (16) are portions that align with the plurality of cavities (12) formed on the housing (10A); and
the plurality of cavities (12) comprising of a central cavity for receiving a sample wherein the sample is deposited on the tray (10B) at an area overlapping with the central cavity to make way to the designated reaction zones (16) to identify and quantify a specific biomarker present in the sample.

2. The diagnostic device (10) as claimed in claim 1, wherein the designated reaction zone (16) is deposited with chemicals in an effective concentration and volume on tray (10B) wherein the designated reaction zone (16) corresponds to a specific biomarker wherein raw intensity values are directly proportional to the concentration of the specific biomarker.

3. The diagnostic device (10) as claimed in claim 1, wherein the housing (10A) preferably comprises of six cavities for measuring six biomarkers in a urine sample, more preferably the housing (10A) comprises two cavities for estimation of total protein and creatinine wherein the plurality of cavities (12), design, and the tray (10B) are varied depending on the specific biomarkers required to be tested.

4. The diagnostic device (10) as claimed in claim 1, wherein the diagnostic device (10) additionally comprises an image capturing device to capture a colour change on the corresponding designated reaction zone (16) and an image processing unit to process an image to identify the colour change in the designated reaction zone (16) due to the specific biomarkers including but not limited to total proteins, creatinine, uric acid, nitrite test, leukocyte esterase (LE), and phosphates.

5. The diagnostic device (10) as claimed in claim 1, wherein the diagnostic device (10) additionally comprises a data interpretation unit to compute quantitative values depending upon an intensity of colour of the designated reaction zone (16) wherein a data is computed using artificial intelligence (AI) and at least one machine learning (ML) algorithms.

6. The diagnostic device (10) as claimed in claim 5, wherein a report is generated and displayed for health conditions including but not limited for kidney, liver, heart, and any other organ function using the specific biomarkers not only in human samples but also from any animal species samples with a sensitivity and specificity of 85-90% and an accuracy of 80-99%.

7. The diagnostic device (10) as claimed in claim 1, wherein the diagnostic device (10) is configured to perform different kinds of blood tests and for detection of any biomarker from any sample material including but not limited to water, urine, blood, sputum, cerebrospinal fluid, bone marrow, milk, or any cancer biomarkers.

8. A process for identifying and quantifying a specific biomarker using a diagnostic device (10), said process comprising the steps of:
a. depositing a sample on a tray (10B) which is a filter paper printed with a hydrophobic pattern (14) to limit the water or other chemicals flow out of a designated reaction zone (16);
b. placing the tray (10B) inside a housing (10A) having plurality of cavities (12) including but not limited to at least six cavities;
c. depositing chemicals on the designated reaction zones (16) in appropriate concentration and volume on the tray (10B) which is dried up for few minutes;
d. identifying the specific biomarkers on each designated reaction zone (16) after depositing the sample, reactions take place at the designated reaction zone (16) and once a user tests positive for any of the specific biomarker, a colour change takes place on the corresponding designated reaction zone (16);
e. capturing of an image by an image capturing device and processing of the captured image by a processing unit to identify change of colour in the designated reaction zone (16) due to the specific biomarkers such as total proteins, creatinine, uric acid, nitrite test, leukocyte esterase (LE), and phosphates;
f. processing of the captured images using a data interpretation unit to compute quantitative values depending on change in intensity of colour of the designated reaction zone (16); and
g. generating and displaying a report for health conditions including but not limited to kidney, liver, heart, and any other organ function using the specific biomarkers not only in human samples but also from any animal species samples with a sensitivity and specificity of 85-90% and an accuracy of 80-99%.

9. The process for identifying and quantifying the specific biomarker as claimed in claim 8, wherein a cloud-based server stores a data which is computed using artificial intelligence (AI) and at least one machine learning (ML) algorithms for generating the report.

10. The process for identifying and quantifying the specific biomarker as claimed in claim 8, wherein said process is configured to perform different kinds of blood tests and for detection of any biomarker from any sample material including but not limited to water, urine, blood, sputum, cerebrospinal fluid, bone marrow, milk, or any cancer biomarkers
